(19) United States
(12) Patent Application Publication
(10) Pub. No.: US 2004/0063090 A1
(43) Pub. Date: Apr. 1, 2004

(54) Method for quantification of recombinant viruses

(76) Inventor: Pier Paolo Claudio, Philadelphia, PA (US)

Correspondence Address: Louis M Heidelberger, Reed Smith, 2500 One Liberty Place, 1650 Market Street, Philadelphia, PA, 19103, US

(21) Appl. No.: 10/250,805

(22) PCT Filed: Jan. 7, 2002
(86) PCT No.: PCT/US02/00247

(30) Foreign Application Priority Data
Jan. 5, 2001 (US) 60259972

(51) Int. Cl. C12Q 1/70; C07H 21/04

(52) U.S. Cl. 435/5; 536/23.72

(57) ABSTRACT

Titration is an important and critical step in dosing recombinant virus for gene therapy. A relatively fast, convenient and sensitive method that allows for precise quantification of recombinant retrovirus is presented. The method is based on PCR amplification of a foreign gene by the PRINS (primer in situ DNA synthesis) technique. The PRINS technique is based on the sequence-specific annealing of unlabeled oligonucleotide DNA in situ. This oligonucleotide operates as a primer for in situ chain elongation catalyzed by the Taq I polymerase. Using -labeled nucleotides as a substrate for chain elongation, the neo-synthetic DNA is labeled by an FITC-conjugated anti-antibody. To avoid the possibility of false positives, the puromycin resistance gene, which is associated with the transgene in the same viral vector and is not normally present in mammalian cells was amplified. The retroviral titer was evaluated by counting FITC-positive cells after PRINS labeling, while knowing the number of cells that were transduced with different amounts of viral supernatant. A comparable viral concentration of 1×10⁷ infectious units/mL was found among the retroviruses.

### BACKGROUND OF THE INVENTION

Genetic transfer is a rapidly developing field in modem medicine which offers the prospect of providing therapies against many acquired diseases such as cancer, neuro-degenerative disorders, and hereditary diseases.

This new approach of therapy could in the future replace the conventional pharmaceutical and medical treatment and allow for the permanent correction of dysfunctional or cancerous cells. There are many gene delivery systems that have been designed to introduce either DNA or oligonucleotides in mammalian cells. They comprise viral-based systems and other systems such as cationic liposomes and receptor-mediated polylisine-DNA complexes. Some of these viral systems are well-characterized for gene therapy use, such as Retroviruses, Adenoviruses, Adeno-Associated virus and now SV40 viruses, while others are still not very well-known. Romano, G., Claudio, P. P., Kaiser, H. E., and Giordano, A. (1998) Recent advances, prospects and problems in designing new strategies for oligonucleotide and gene delivery in therapy In Vivo 12, 59-67; Strayer, D. S., Duan, L. X., Ozaki, I., Milano, J., Bobraski, L. E., and Bagasra, O. (1997) Titering replication-defective virus for use in gene transfer Biotechniques 22, 447-50. Each of these systems has advantages and disadvantages; however, none of these methods is without potential problems that could compromise cells' viability such as cell killing and toxicity, or technical problems such as lack of transduction efficiency and lack of a prolonged expression in target cells. Romano, G., Claudio, P. P., Kaiser, H. E., and Giordano, A. (1998) Recent advances, prospects and problems in designing new strategies for oligonucleotide and gene delivery in therapy In Vivo 12, 59-67

One of the methods used to titer recombinant retrovirus is to perform colony assays, by using drugs to select a known number of cells that have been transduced with different amounts of viral supernatant. By the number of colonies counted, one can retrospectively identify the viral titer and therefore define the viral quantity by pfu/ml (plaque forming unit/milliliter). The greatest problem in tittering recombinant retroviruses with this method is that if the transgene delivered is a tumor suppressor gene (a gene that blocks cell growth when overexpressed) the number of colonies counted might not correspond to the real titer of the recombinant virus. We encountered this problem in our attempts to titer retroviruses carrying a gene called RB2/p130, a member of the retinoblastoma family whose enhanced expression results in growth arrest of cells in the G1 phase of the cell cycle as well as regression of tumor xenograft in mice. Claudio, P. P., Howard, C. M., Baldi, A., De Luca, A., Fu, Y., Condorelli, G., Sun, Y., Colburn, N., Calabretta, B., and Giordano, A. (1994) p130/pRb2 has growth suppressive properties similar to yet distinctive from those of retinoblastoma family members pRb and p107 Cancer Res 54, 5556-60; Claudio, P. P., De Luca, A., Howard, C. M., Baldi, A., Firpo, E. J., Koff, A., Paggi, M. G., and Giordano, A. (1996) Functional analysis of pRb2/p130 interaction with cyclins Cancer Res 56, 2003-8; Howard, C. M., Claudio, P. P., Gallia, G. L., Gordon, J., Giordano, G. G., Hauck, W. W., Khalili, K., and Giordano, A. (1998) Retinoblastoma-related protein pRb2/p130 and suppression of tumor growth in vivo [see comments] J Natl Cancer Inst 90, 1451-60; Claudio, P. P., Fratta, L., Farina, F., Howard, C. M., Stassi, G., Numata, S., Pacilio, C., Davis, A., Lavitrano, M., Volpe, M., Wilson, J. M., Trimarco, B., Giordano, A., and Condorelli, G. (1999) Adenoviral RB2/p130 Gene Transfer Inhibits Smooth Muscle Cell Proliferation and Prevents Restenosis After Angioplasty Circ Res. 85, 1032-1039; Claudio, P. P., Howard, C. M., Pacilio, C., Cinti, C., Romano, G., Minimo, C., Maraldi, N. M., Minna, J. D., Gelbert, L., Micheli, P., Leoncini, L., Tosi, G. M., Micheli, P., Caputi, M., Giordano, G. G., and Giordano, A. (2000) Suppression of tumor growth in vivo by retroviral-mediated Retinoblastoma-related RB2/p130 gene transfer Cancer Research 60, 2737-44.

To solve the aforementioned problem, the retroviral titer was evaluated by counting FITC-positive cells after PRINS (primer in situ DNA synthesis) labeling. This method is largely used to identify single copies of a gene, repeat sequences in cytogenetic studies or aneuploidy and chromosomal heterogeneity in tumoral cells. Cinti, C., Santi, S., and Maraldi, N. M. (1993) Localization of single copy gene by PRINS technique Nucleic Acids Res 21, 5799-800; Koch, J., Hindkjaer, J., Kolvraa, S., and Bolund, L. (1995) Construction of a panel of chromosome-specific oligonucleotide probes (PRINS-primers) useful for the identification of individual human chromosomes in situ Cytogenet Cell Genet 71, 142-7; Koch, J. (1996) Primed in Situ Labeling as a Fast and Sensitive Method for the Detection of Specific DNA Sequences in Chromosomes and Nuclei Methods 9, 122-8; Hindkjaer, J., Koch, J., Mogensen, J., Kolvraa, S., and Bolund, L. (1994) Primed in situ (PRINS) labeling of DNA Methods Mol Biol 33, 95-107; Hindkjaer, J., Koch, J., Brandt, C., Kolvraa, S., and Bolund, L. (1996) Primed in situ labeling (PRINS). A fast method for in situ labeling of nucleic acids Mol Biotechnol 6, 201-11; Velagaleti, G. V., Tharapel, S. A., Martens, P. R., and Tharapel, A. T. (1997) Rapid identification of marker chromosomes using primed in situ labeling (PRINS) Am J Med Genet 71, 130-3; Pellestor, F., Andreo, B., and Coullin, P. (1998) Assessment of chromosomal heterogeneity in tumoral cell lines using PRINS technique Ann Genet 41, 141-8; Pellestor, F., Andreo, B., and Coullin, P. (1999) Interphasic analysis of aneuploidy in cancer cell lines using primed in situ labeling Cancer Genet Cytogenet 111, 111-8. It is a fast, simple, and sensitive method that has a wide range of application in clinical cytogenetics.

### BRIEF DESCRIPTION OF DRAWINGS

FIG. 1. Primer in situ DNA synthesis (PRINS) of the puromycin-resistance gene on H23 cells transduced with 1000 μL of retroviral supernatant carrying the puromycin-resistance gene alone (A) or in combination with the RB2/p130 gene in either the sense (B) or the antisense orientation (C) or empty retroviral particles containing only gag/pol and env as a negative control (D). 1000× final magnification. Bar=10 μm.

FIG. 2 A) Hematoxylin and eosin (H-E) staining of an H23 tumor grown in nude mice transduced with the retroviruses carrying the puromycin-resistance gene (Pac) showing features of a poorly differentiated adenocarcinoma (1000× final magnification); and Primer in situ DNA synthesis (PRINS) using primers that amplify a 425-nucleotide fragment of the puromycin-resistance gene present in the Pac, and RB2/p130 retroviruses in OTC frozen sections. B) Tumor samples from H23 tumors transduced with empty virus as a negative control. C) Tumor samples from H23 tumors transduced with retroviruses carrying the puromycin-resistance gene alone. D) Tumor samples from H23 tumors transduced with retrovirus carrying the RB2/p130 gene. Bar=10 μm.

### DETAILED DESCRIPTION OF THE INVENTION

The PRINS technique is based on the sequence-specific annealing of unlabeled oligonucleotide DNA in situ. This oligonucleotides operates as a primer for in situ chain elongation catalyzed by the Taq I polymerase. Using digoxigenin-labeled nucleotides as a substrate for chain elongation, the neo-synthetic DNA is labeled by an FITC-conjugated anti-digoxigenin antibody. In order to circumvent the possibility of a false positive, the puromycin-resistance gene, which is included along with RB2/p130 in the same viral vector and is not normally present in mammalian cells was amplified. Additionally, in order to avoid background due to a specific binding of FITC-conjugated antibody, nucleotides labeled with synthetic, which is not normally present in biological samples were used.

Retroviral-mediated gene transfer of the putative tumor suppressor gene RB2/p130 in lung cancer cells was carried out with an MLV-based system. Soneoka, Y., Cannon, P. M., Ramsdale, E. E., Griffiths, J. C., Romano, G., Kingsman, S. M., and Kingsman, A. J. (1995) A transient three-plasmid expression system for the production of high titer retroviral vectors Nucleic Acids Res 23, 628-33; Pear, W. S., Nolan, G. P., Scott, M. L., and Baltimore, D. (1993) Production of high-titer helper-free retroviruses by transient transfection Proc Natl Acad Sci USA 90, 8392-6; Landau, N. R., and Littman, D. R. (1992) Packaging system for rapid production of murine leukemia virus vectors with variable tropism J Virol 66, 5110-3; Romano, G., Guan, M., Long, W. K., and Henderson, E. E. (1997) Differential effects on HIV-1 gene regulation by EBV in T lymphocytic and promonocytic cells transduced to express recombinant human CR2 Virology 237, 23-32. This system allows for the production of high-titer retroviral stocks by transient transfection of human renal carcinoma 293T/17 cells. This cell line is highly transfectable and expresses the SV40 large T antigen, Pear, W. S., Nolan, G. P., Scott, M. L., and Baltimore, D. (1993) Production of high-titer helper-free retroviruses by transient transfection Proc Natl Acad Sci USA 90, 8392-6. The packaging components gag-pol and env of the MLV are placed on two different plasmids that contain the SV40 origin of replication in their backbone. For this reason, these plasmids will be amplified after being transfected in 293T/17 cells by the SV40 large T antigen. In addition, the MLV packaging components are under the control of the strong human cytomegalovirus immediate early promoter (hCMVi.e.) Soneoka, Y., Cannon, P. M., Ramsdale, E. E., Griffiths, J. C., Romano, G., Kingsman, S. M., and Kingsman, A. J. (1995) A transient three-plasmid expression system for the production of high titer retroviral vectors Nucleic Acids Res 23, 628-33. These two features result in an overexpression system that allows for a rapid generation of high-titer helper-virus-free retroviral stocks, which is a critical requirement for efficient transduction of target cells. The RB2/p130 ORF was placed in MSCV-based transfer vectors Hawley, R. G., Lieu, F. H., Fong, A. Z., and Hawley, T. S. (1994) Versatile retroviral vectors for potential use in gene therapy Gene Ther 1, 136-8; Hawley, R. G., Lieu, F. H., Fong, A. Z., and Hawley, T. S. (1994) Versatile retroviral vectors for potential use in gene therapy Gene Ther 1, 136-8 which contain genetically modified 5′ long terminal repeats (LTRs), to achieve both high levels and long-term expression of the transgene.

Retroviral titer was determined by counting the FITC-positive cells obtained after PRINS using primers that amplify a 425-nt fragment of the puromycin-resistance gene present in the plasmids MSCVPac and MSCVPac pRB2/p130, in the sense or the antisense orientation, that were transduced.

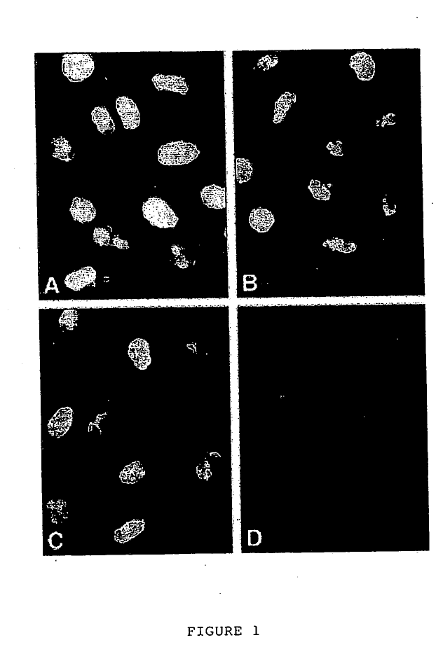

Four sets of samples of human H23 and A549 human lung adenocarcinoma cells were prepared for the PRINS reaction. Cells were transduced with 20 μL, 50 μL, 100 μL or 1 mL of supernatant-containing retroviruses carrying the puromycin-resistance gene alone or in combination with the RB2/p130 gene in the sense or the antisense orientation. FIG. 1 depicts a representative example of the results obtained by transducing the H23 cells with 1000 μL of the different retroviral supernatant. Supernatant collected from a cotransfection of 293T/17 cells with only the plasmids carrying gag/pol and env (empty retroviral vector) was used as a negative control. FITC-positive cells were counted and photographed under a confocal microscope. Ten fields at 400× magnification were randomly chosen in each slide and scored, and FITC-positive cells were counted. A comparable viral concentration of 1×10⁷ infectious units/mL was found among the retroviruses (Table 1).

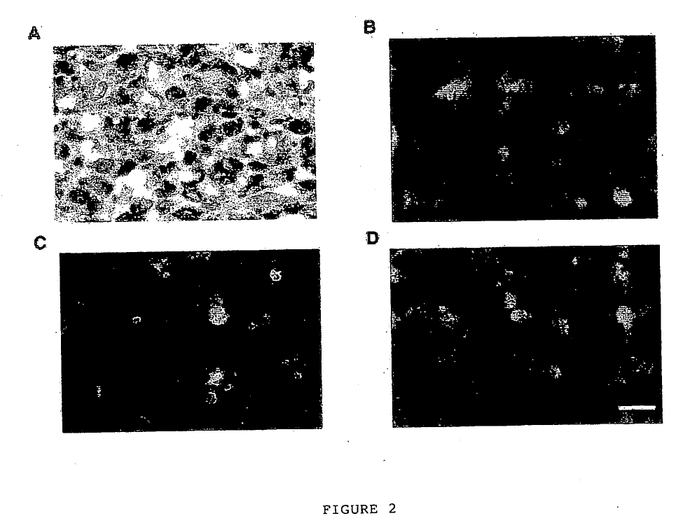

The same process was applied to OCT tumor embedded (Sakura Finetek USA, Inc. Torrance, Calif.) frozen sections of H23 cells grown in nude mice. For this purpose, we chose to use frozen sections and not formalin-fixed and paraffin-embedded tissues because this treatment may subject the specimens to DNA damages, thus limiting the in vivo aspect of this technique. Additionally, since paraffin is autofluorescent, this histological treatment is not suitable for immunofluorescent studies. Transduction in vivo was confirmed by the PRINS technique on OTC frozen sections of H23 cell tumor graft transduced with viruses carrying RB2/p130 in either the sense or antisense orientation or with the control Pac retrovirus and, as a negative control, with empty virus or non-transduced tumors. The tumors transduced in vivo with the Pac, and RB2/p130 retroviruses were positive for amplification of the puromycin-resistance fragment (FIG. 2 C, D), while the tumors transduced with empty retroviral vector and non-transduced tumors were negative (FIG. 2 B and data not shown). Taken together, these results demonstrate that the PRINS technique, used to determine viral titers as well as viral transduction efficiency, opens the way to new strategies to test the efficiency of virally based gene delivery systems before their application in genetic therapy.

Plasmids

The plasmids pHIT60 (CMV-MLV-gag-pol-SV40ori; where CMV is cytomegalovirus, MLV is murine leukemia virus, gag is capsid proteins, pol is reverse transcriptase and integrase, SV40 is simian virus 40, and ori is origin), pHIT110 (CMV-Neo-SV40ori; where neo is neomycin resistance gene), pHIT111 (CMV-lacZ-SV40 promoter-Neo-SV40ori; where lacZ is the E. coli β-galactosidase gene) and pHIT456 (CMV-MLV-amphotropic env-SV40ori; where env is envelope proteins) have been previously described. Soneoka, Y., Cannon, P. M., Ramsdale, E. E., Griffiths, J. C., Romano, G., Kingsman, S. M., and Kingsman, A. J. (1995) A transient three-plasmid expression system for the production of high titer retroviral vectors Nucleic Acids Res 23, 628-33. The plasmids MSCVneoEB and MSCVPac (derived from the murine stem cell virus [MSCV] and LN retroviral vectors, where neo is the neomycin an Hawley, R. G., Lieu, F. H., Fong, A. Z., and Hawley, T. S. (1994) Versatile retroviral vectors for potential use in gene therapy Gene Ther 1, 136-8d pac is the puromycin resistance gene) have been previously described. The full-length cDNA sequence of RB2/p130 was subcloned into the retroviral vectors MSCVneoEB and MSCVPac in the sense and antisense orientations. Claudio, P. P., Howard, C. M., Pacilio, C., Cinti, C., Romano, G., Minimo, C., Maraldi, N. M., Minna, J. D., Gelbert, L., Micheli, P., Leoncini, L., Tosi, G. M., Micheli, P., Caputi, M., Giordano, G. G., and Giordano, A. (2000) Suppression of tumor growth in vivo by retroviral-mediated Retinoblastoma-related RB2/p130 gene transfer Cancer Research 60, 2737-44

Cell Lines

The human lung adenocarcinoma cell line H23 has been previously described Wawrzynczak, E. J., Derbyshire, E. J., Henry, R. V., Parnell, G. D., Smith, A., Waibel, R., and Stahel, R. A. (1990) Selective cytotoxic effects of a ricin A chain immunotoxin made with the monoclonal antibody SWA11 recognizing a human small cell lung cancer antigen Br J Cancer 62, 410-4. The cell line A549 (human lung carcinoma) was purchased from the American Type Culture Collection (Manassas, Va.). The 293T/17 cell line (human renal carcinoma) Pear, W. S., Nolan, G. P., Scott, M. L., and Baltimore, D. (1993) Production of high-titer helper-free retroviruses by transient transfection Proc Natl Acad Sci USA 90, 8392-6 was purchased from the American Type Culture Collection upon authorization of Rockefeller University. A549 and H23 cells were maintained in Dulbecco's modified Eagle medium (D-MEM) supplemented with 10% fetal bovine serum (FBS), 2 mM 1-glutamine. The 293T/17 cell line was maintained in DMEM supplemented with 10% heat inactivated FBS and 2 mM 1-glutamine.

Recombinant Retrovirus Preparation, Transduction and Determination of Viral Titer

The preparation of recombinant retrovirus has been previously described Claudio, P. P., Howard, C. M., Pacilio, C., Cinti, C., Romano, G., Minimo, C., Maraldi, N. M., Minna, J. D., Gelbert, L., Micheli, P., Leoncini, L., Tosi, G. M., Micheli, P., Caputi, M., Giordano, G. G., and Giordano, A. (2000) Suppression of tumor growth in vivo by retroviral-mediated Retinoblastoma-related RB2/p130 gene transfer Cancer Research 60, 2737-44; Cinti, C., Santi, S., and Maraldi, N. M. (1993) Localization of single copy gene by PRINS technique Nucleic Acids Res 21, 5799-800; Koch, J., Hindkjaer, J., Kolvraa, S., and Bolund, L. (1995) Construction of a panel of chromosome-specific oligonucleotide probes (PRINS-primers) useful for the identification of individual human chromosomes in situ Cytogenet Cell Genet 71, 142-7; Koch, J. (1996) Primed in Situ Labeling as a Fast and Sensitive Method for the Detection of Specific DNA Sequences in Chromosomes and Nuclei Methods 9, 122-8; Hindkjaer, J., Koch, J., Mogensen, J., Kolvraa, S., and Bolund, L. (1994) Primed in situ (PRINS) labeling of DNA Methods Mol Biol 33, 95-107; Hindkjaer, J., Koch, J., Brandt, C., Kolvraa, S., and Bolund, L. (1996) Primed in situ labeling (PRINS). A fast method for in situ labeling of nucleic acids Mol Biotechnol 6, 201-11; Velagaleti, G. V., Tharapel, S. A., Martens, P. R., and Tharapel, A. T. (1997) Rapid identification of marker chromosomes using primed in situ labeling (PRINS) Am J Med Genet 71, 130-3; Pellestor, F., Andreo, B., and Coullin, P. (1998) Assessment of chromosomal heterogeneity in tumoral cell lines using PRINS technique Ann Genet 41, 141-8; Pellestor, F., Andreo, B., and Coullin, P. (1999) Interphasic analysis of aneuploidy in cancer cell lines using primed in situ labeling Cancer Genet Cytogenet 111, 111-8; Soneoka, Y., Cannon, P. M., Ramsdale, E. E., Griffiths, J. C., Romano, G., Kingsman, S. M., and Kingsman, A. J. (1995) A transient three-plasmid expression system for the production of high titer retroviral vectors Nucleic Acids Res 23, 628-33. Briefly, the packaging components gag-pol and env of the MLV are placed on two different plasmids that contain the SV40 origin of replication in their backbone. The MLV packaging components are under the control of the strong human cytomegalovirus immediate early promoter (hCMVi.e.) Soneoka, Y., Cannon, P. M., Ramsdale, E. E., Griffiths, J. C., Romano, G., Kingsman, S. M., and Kingsman, A. J. (1995) A transient three-plasmid expression system for the production of high titer retroviral vectors Nucleic Acids Res 23, 628-33. These two features result in an overexpression system that allows for a rapid generation of high-titer helper-virus-free retroviral stocks, which is a critical requirement for efficient transduction of target cells. The RB2/p130 ORF was placed in the MSCV-based transfer vectors. Hawley, R. G., Lieu, F. H., Fong, A. Z., and Hawley, T. S. (1994) Versatile retroviral vectors for potential use in gene therapy Hawley, R. G., Lieu, F. H., Fong, A. Z., and Hawley, T. S. (1994) Versatile retroviral vectors for potential use in gene therapy Gene Ther 1, 136-8; Gene Ther 1, 136-8 Hawley, R. G., Lieu, F. H., Fong, A. Z., and Hawley, T. S. (1994) Versatile retroviral vectors for potential use in gene therapy Gene Ther 1, 136-8, which contain genetically modified 5′ long terminal repeats (LTRs), to achieve both high levels and long-term expression of the transgene. Calcium phosphate transient co-transfection Soneoka, Y., Cannon, P. M., Ramsdale, E. E., Griffiths, J. C., Romano, G., Kingsman, S. M., and Kingsman, A. J. (1995) A transient three-plasmid expression system for the production of high titer retroviral vectors Nucleic Acids Res 23, 628-33; Hawley, R. G., Lieu, F. H., Fong, A. Z., and Hawley, T. S. (1994) Versatile retroviral vectors for potential use in gene therapy Gene Ther 1, 136-8; Pear, W. S., Nolan, G. P., Scott, M. L., and Baltimore, D. (1993) Production of high-titer helper-free retroviruses by transient transfection Proc Natl Acad Sci USA 90, 8392-6; Graham, F. L., and Eb, A. J. v. d. (1973) Transformation of rat cells by DNA of human adenovirus 5 Virology 54, 536-9 of the three plasmids together [pHIT60 (CMV-MLV-gag-pol-SV40ori)], [pHIT456 (CMV-MLV-amphotropic env-SV40ori)] and MSCVPac-RB2/p130 in the 293T/17 result in the production of retrovirus in the cellular supernatant. The retroviral supernatant was collected 48 hours after transfection and filtered through a 0.45 μm (pore size) filter. Viral concentration was measured by transducing H23 and A549 human lung adenocarcinoma cell lines. Viral titers were determined by counting the fluorescein isothiocyanate (FITC)-positive cells after PRINS labeling.

Animal Studies

Animal care and human use and treatment of mice were in strict compliance with the following: 1) institutional guidelines, 2) the Guide for the Care and Use of Laboratory Animals (National Academy of Sciences, 1996), and 3) the Association for Assessment and Accreditation of Laboratory Animal Care International. Tumors were generated by the subcutaneous injection of 2.5×10⁶ H23 into each flank of nude mice (female NU/NU-nuBR outbred, isolator-maintained mice, 4-5 weeks old from Charles Rivers Wilmington, Mass.), as previously described Claudio, P. P., Howard, C. M., Pacilio, C., Cinti, C., Romano, G., Minimo, C., Maraldi, N. M., Minna, J. D., Gelbert, L., Micheli, P., Leoncini, L., Tosi, G. M., Micheli, P., Caputi, M., Giordano, G. G., and Giordano, A. (2000) Suppression of tumor growth in vivo by retroviral-mediated Retinoblastoma-related RB2/p130 gene transfer Cancer Research 60, 2737-44.

When the tumors reached a volume of approximately 20 mm³ after 15 days, each tumor was transduced with 5×10⁶ retroviruses carrying the Pac gene alone or the Pac gene and RB2/p130 open reading frame (ORF), with three animals per group, by direct injection of 20 μL of retroviral supernatant directly into each of the tumors.

Animals were sacrificed by CO₂ asphyxiation when Pac and LacZ retroviral-transduced tumors reached a size of 300-350 mm³. Tissues to be sectioned were placed in OTC (Sakura Finetek USA, Inc., Torrance, Calif.), frozen in liquid nitrogen, and stored at −80° C. or preserved in neutral-buffered formalin at 4° C. before embedding in paraffin.

PRINS

Four samples of A549 and H23 cells were prepared for the PRINS reaction Cinti, C., Santi, S., and Maraldi, N. M. (1993) Localization of single copy gene by PRINS technique Nucleic Acids Res 21, 5799-800. Cells were plated on slides at a concentration of 5×10⁵ cells per dish in 10 mL of DMEM and 2% of heat inactivated Fetal Bovine Serum, and transduced with 20 μL, 50 μL, 100 μL, or 1 mL of retroviruses carrying the puromycin-resistance gene alone or in combination with the Rb2/p130 ORF in the sense or the antisense orientation. As a negative control, cells were transduced with supernatant collected from a co-transfection of 293T/17 cells with only the plasmids carrying gag/pol and env genes (empty viruses).

Samples were fixed in pre-cooled methanol and glacial acetic acid 3:1 (vol:vol) for 10 minutes at room temperature and air dried for 12-24 hours. The next day the samples were dehydrated in a series of ethanol solutions (70%, 80% and 100%) each for 5 minutes at 4° C. and air-dried.

For the PRINS reaction, the primers PUR5 (5′-TCACCGAGCTGCAAGAAC-3′) and PUR3 (5′-GTCCTTCGGGCACCTCGA-3′) were used to amplify a stretch of 425 bp in the puromycin-resistance gene present in the plasmids MSCVPac and MSCVPac pRb2/p130, both sense and antisense. Each sample was incubated with 50 μL of the reaction mixture at 95° C. (denaturation) for 5 minutes, at 58° C. (annealing) for 30 minutes, and at 74° C. (elongation) for 90 minutes. The reaction mixture contained 100 ng of each primer, 1×PCR buffer with 1.5 mM MgCl₂ (QIAGEN, Valencia, Calif.), 1 mM deoxyadenosine 5′ triphosphate, 1 mM deoxycytosine 5′ triphosphate, 1 mM deoxyguanosine 5′ triphosphate, 0.65 mM deoxythymidine 5′ triphosphate, 0.35 mM -11-deoxyuridine 5′ triphosphate, alkali-labile, (Boheringer Mannheim, Indianapolis, Ind.), and 2.5 units of Taq DNA polymerase (QIAGEN, Valencia, Calif.).

The reaction mixture was deposited on each slide and topped with a cover slide which was sealed with rubber cement glue. After elongation at 74° C., the slides were washed twice for 10 minutes in 2×SSC at room temperature to remove excess reaction mixture. The slides were then incubated in 2×SSC containing 2% bovine serum albumin (BSA) for 10 minutes at room temperature. Monoclonal FITC-conjugated anti-digoxigenin-antibody (Boheringer Mannheim, Indianapolis, Ind.) was diluted 1:200 into 2×SSC and 2% bovine serum albumin and used for detection of digoxigenin-11-deoxyuridine 5′ triphosphate incorporation. The slides were incubated with the FITC-conjugated anti-digoxigenin antibody for 30 minutes at room temperature in a dark humid chamber and then washed twice for 5 minutes in 2×SSC to remove excess antibody. The samples were also treated with a solution of propidium iodide 1 μg/mL (Sigma St. Louis, Mo.) to stain the unlabeled DNA and then washed in water. Slides were then observed and stained cells were counted and photographed under a confocal microscope. The same process was applied to OCT tumor embedded (Sakura Finetek USA, Inc. Torrance, Calif.) frozen sections of H23 cells grown in nude mice.

TABLE 1
Quantitative data regarding the percentage and standard deviation
(S.D.) of FITC positive cells at the PRINS reaction. A viral titer of 1 ×
10⁷ was calculated with the following formula: {[(Number plated cells) ×
(% FITC positive cells)/100]/(Number of mL of retroviral supernatant used
to transduce)} × 10 mL.

| | | | MSCV | | MSCV | |
|---|---|---|---|---|---|---|
| | MSCV Pac | | Pac pRb2/p130 S | | Pac pRb2/p130 AS | |
| | % (+cells) | ±S.D. | % (+cells) | ±S.D. | % (+cells) | ±S.D. |
| 20 μL | 4.7 | 1.7 | 3.8 | 1.6 | 3.6 | 1.4 |
| 50 μL | 13.7 | 2.45 | 13.2 | 1.8 | 13 | 2.3 |
| 100 μL | 29.3 | 4 | 27.1 | 2.8 | 27.8 | 3.1 |
| 1000 μL | 100 | 0 | 100 | 0 | 100 | 0 |

All publications and references, including but not limited to patent applications, cited in this specification, are herein incorporated by reference in their entirety as if each individual publication or reference were specifically and individually indicated to be incorporated by reference herein as being fully set forth.

The previous description of the preferred embodiments is provided to enable any person skilled in the art to make and use the present invention. The various modifications to these embodiments will be readily apparent to those skilled in the art, and the generic principles defined herein may be applied to other embodiments without the use of the inventive faculty. Thus, the present invention is not intended to be limited to the embodiments shown herein but is to be accorded the widest scope consistent with the principles and novel features disclosed herein. Accordingly, this invention includes all modifications encompassed within the spirit and scope of the invention as defined by the claims.

What is claimed is:

1. A method for determining titers of a virus in cells infected with the virus, the method comprising:
(a) adding to said cells a primer extension reaction mixture, wherein the primer extension reaction mixture includes a nucleotide oligo primer specific to a nucleic acid segment of the virus, and a digoxigenin labeled nucleotide;
(b) incubating said cells under conditions sufficient to facilitate the oligo primer extension;
(c) adding a fluorescent molecule conjugated anti-digoxigenin antibody to said cells after step (b); and
(d) counting the fluorescent positive cells and determining titers of the virus from the fluorescent positive cell counts.

2. The method of claim 1, wherein the virus is a recombinant virus.

3. The method of claim 2, wherein the recombinant virus is a recombinant retrovirus.

4. The method of claim 2, wherein the recombinant virus is a recombinant adenovirus.

5. The method of claim 1, wherein the cells are tumor or cancer cells.

6. The method of claim 5, wherein the cells include human glioblastoma cells.

7. The method of claim 5, wherein the cells include melanoma cells.

8. The method of claim 5, wherein the cells include breast cancer cells.

9. The method of claim 5, wherein the cells include lung cancer cells.

10. The method of claim 5, wherein the cells include endometrial cancer cells.

11. The method of claim 5, wherein the cells include stomach carcinoma cells.

12. A method for determining titers of a virus in cells infected with the virus, the method comprising:
(a) adding to said cells a primer extension reaction mixture, wherein the primer extension reaction mixture includes an oligo primer specific to a nucleic acid segment of the virus and at least one fluorescent molecule labeled nucleotide;
(b) incubating said cells under conditions sufficient to facilitate the oligo primer extension; and
(d) counting the fluorescent positive cells and determining titers of the virus from the fluorescent positive cell counts.
